(19) United States
(12) Patent Application Publication
(10) Pub. No.: US 2024/0238401 A1
(43) Pub. Date: Jul. 18, 2024

(54) SOLID COMPOSITION, FREEZE-DRYING METHOD AND GLASS VIAL

(71) Applicant: Takeda Vaccines, Inc., Cambridge, MA (US)

(72) Inventors: Tuhin BHOWMIK, Leominster, MA (US); Ke GONG, Lexington, MA (US); Zhengrong CUI, Austin, TX (US); Robert O. WILLIAMS, Austin, TX (US); Haiyue XU, Austin, TX (US)

(21) Appl. No.: 18/561,848

(22) PCT Filed: May 23, 2022
(86) PCT No.: PCT/US2022/030550

§ 371 Date: Nov. 17, 2023

Related U.S. Application Data
(60) Provisional application No. 63/191,713, filed on May 21, 2021.

(51) Int. Cl. A61K 39/125; A61J 1/06; A61K 9/16; A61K 9/19; A61K 39/00

(52) U.S. Cl. CPC A61K 39/125; A61K 9/1623; A61K 9/19; A61J 1/065; A61K 2039/5258; A61K 2039/55505

(57) ABSTRACT

The present invention is directed to a solid composition comprising: antigen particles comprising virus like particles (VLPs) adsorbed on adjuvant particles.

### CROSS-REFERENCE TO RELATED APPLICATION

This application claims the benefit of priority to U.S. Provisional Application No. 63/191,713, filed May 21, 2021, the disclosure of which is hereby incorporated by reference in its entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: LIGO_029_01 WO_SeqList_ST25.txt, date recorded May 23, 2021, file size 14,251 bytes).

### FIELD OF THE INVENTION

The present invention relates to a solid composition, in particular to an immunogenic composition, and a freeze-drying method for removing water from an aqueous suspension, and a glass vial containing an amount of a Norovirus vaccine in solid form.

### BACKGROUND OF THE INVENTION

The World Health Organization (WHO) recommends that the majority of the human vaccines should be kept in cold chain storage of 2 to 8° C. during transport and storage (see Temperature sensitivity of vaccines. Geneva: World Health Organization; 2006). Many vaccines such as Prevnar-13 and Gardasil-9 contain insoluble aluminum salts as adjuvants. Unfortunately, vaccines adjuvanted with aluminum salts must not be exposed to freezing temperatures during transport or storage, because slow freezing causes irreversible aggregation of the insoluble aluminum salt microparticles, leading to a loss of vaccine potency and efficacy (see Zapata M I et al., Journal of pharmaceutical sciences. 1984; 73(1):3-8; Kristensen D et al., Vaccine. 2011; 29(41): 7122-4; Diminsky D et al., Vaccine. 1999;18(1-2):3-17; and Boros C A et al., Vaccine. 2001;19(25-26):3537-4). Conventional freeze-drying techniques are not sufficiently preventing irreversible aggregation of the insoluble aluminum salt microparticles and, therefore, loss of vaccine potency and efficacy due to the relatively slow freezing rate. In addition, the protein antigens in the liquid vaccines are too fragile to be stable when exposed for an extended period of time to room temperature or above.

The cold chain requirement significantly hinders the distribution of vaccines globally because it brings costly waste to the vaccine supply (see Wirkas T et al., Vaccine. 2007; 25(4):691-7; and Lydon P et al., Bulletin of the World Health Organization. 2013; 92:86-92), and inadvertent exposure of the vaccines to freezing or heat could compromise their potency, potentially leading to unintentional administration of vaccines with suboptimal potency or efficacy (see Diminsky D et al, Vaccine. 1999; 18(1-2):3-17; Nygaard U C et al., Toxicological Sciences. 2004; 82(2):515-24; Davaalkham D et al., Journal of Epidemiology & Community Health. 2007; 61(7):578-84; Mansoor O and Pillans P, The New Zealand medical journal. 1997; 110(1048):270-2; and Menon P et al., Indian Journal of medical research. 1976; 64(1):25-32).

There is great interest in addressing this problem, and the strategies to solve it are generally two-fold. The first is to add stabilizing reagents in vaccines to prevent aggregation during freezing. For example, the Program for Appropriate Technology (PATH) and its research collaborators have shown that adding glycerol, polyethylene glycol 300, or propylene glycol to vaccines that contain aluminum salts prevents vaccine aggregation and preserves vaccine immunogenicity, even after the vaccines are subjected to multiple exposures to −20° C. (Kristensen D et al., Vaccine. 2011; 29:7122-7124.). Zapata et al. also reported that the adsorption of polymers or surface-active agents, such as hydroxypropyl methylcellulose or polysorbate 80, on aluminum hydroxide prevents aggregation after a freeze-thaw cycle (Zapata M I et al., J. Pharm. Sci. 1984; 73:3-8.). It is thought that the stabilizing agents produce a large steric repulsive region between particles and hinder particle-particle interactions (see Zapata M I et al., J. Pharm. Sci. 1984; 73:3-8.). However, the addition of the aforementioned excipients into a vaccine may result in a more complex formulation and increase the cost per dose of the vaccine.

Another strategy is to convert vaccines into a solid form using freezing and/or drying techniques. However, after conversion to solid form, the vaccines must be reconstituted, and these techniques and their subsequent reconstitution process have been shown to cause particle aggregation and/or significantly alter the immunogenicity of the vaccines.

Norovirus has been indicated as one of the most common causes of acute gastroenteritis in subjects of any age (see Hall A J et al., Taylor & Francis; 2016). It was reported that Norovirus causes nearly 700 million cases of illness with significant morbidity, which leads to a worldwide social burden (i.e., a total of $4.2 billion estimated in direct health system costs and $60.3 billion in societal costs per year) (see Hall A J et al., Taylor & Francis; 2016). More than 200,000 deaths per year are estimated to result from Norovirus illness, primarily in developing countries (Bartsch S M et al., PloS one. 2016; 11(4): e0151219), where a vaccine that does not require cold chain for storage and transport would be most beneficial and essential. In 2016, the WHO stated that the development of a Norovirus vaccine should be an absolute priority. A liquid Norovirus vaccine candidate was developed for intramuscular injection to cover the two genogroups that cause the majority of illness in humans (see Masuda T et al., Open Forum Infectious Diseases; 2018: Oxford University Press; and Bernstein D I et al, The Journal of infectious diseases. 2015; 211(6):870-8). Specifically, it is a bivalent virus like particle (VLP) vaccine and consists of antigens from two Norovirus strains adsorbed on aluminum (oxy)hydroxide in suspension. Data from a phase 1/2 study showed that this vaccine candidate is generally well-tolerated and has a clinically relevant impact on the symptoms and severity of Norovirus illness after challenge (see Masuda T et al., Open Forum Infectious Diseases; 2018: Oxford University Press; and Bernstein D I et al, The Journal of infectious diseases. 2015; 211(6):870-8). However, due to their complex structure, VLP-based vaccines are expected to be unstable, i.e. prone to potency loss with or without adjuvant under various lyophilization conditions. Thus, there is an ongoing and unmet need for providing adjuvanted VLP vaccines, such as Norovirus VLP vaccines, in a form that does not require cold chain for storage and transport while addressing the above-menitoned challenges with respect to particle agglomeration and potency loss of adjuvanted vaccines, in particular of adjuvanted VLP vaccines.

### OBJECTS AND SUMMARY

It is an object of the present invention to provide a composition comprising antigen particles comprising virus like particles (VLPs) adsorbed on adjuvant particles, wherein said composition does not need to be stored at low temperatures, such as 2° C. and 8° C., in order to sufficiently prevent particle aggregation and antigen loss, while preserving the potency of the antigens.

It is a further object of the present invention to obtain from an aqueous suspension including antigen particles comprising virus like particles adsorbed on an adjuvant a solid composition without causing antigen loss or particle aggregation, while preserving the potency of the antigens.

It is a further object of the present invention to obtain a solid composition from an aqueous suspension comprising antigen particles comprising virus like particles adsorbed on an adjuvant, wherein the relative potency is in a range of 50%-150%, including all ranges and subranges therebetween, wherein the relative potency is determined by the potency of the antigen particles of the solid composition relative to the potency of the antigen particles of the aqueous suspension, and wherein the potencies are determined by an in vitro relative potency assay.

It is a further object of the present invention to provide a composition comprising antigen particles comprising virus like particles (VLPs) adsorbed on adjuvant particles, wherein the composition is stable with respect to particle aggregation and the potency of the antigens is preserved under severe conditions, e.g. when exposed to 40° C. and 75% relative humidity, for an extended period of time, such as 4 weeks.

It is a further object of the present invention to provide a composition comprising antigen particles comprising virus like particles (VLPs) adsorbed on adjuvant particles having a low moisture content, in particular less than 3% based on the total mass of said solid composition.

The above objects are achieved by embodiments of the present invention as described and claimed herein.

According to a first aspect, the present invention is therefore directed to a solid composition comprising: antigen particles comprising virus like particles (VLPs) adsorbed on adjuvant particles.

According to a second aspect, the present invention is directed to freeze-drying method for removing water from an aqueous suspension, the aqueous suspension comprising:
antigen particles comprising virus like particles adsorbed on adjuvant particles;
the method comprising the steps:
step 1: providing the aqueous suspension optionally at a temperature ranging from about 2° C. to about 15° C.;
step 2: decreasing the temperature of step 1 with a freezing rate of at least 50 K/s to obtain a first frozen suspension;
step 3: collecting the frozen suspension in a container cooled to the temperature of liquid nitrogen to obtain a second frozen suspension;
step 4: subjecting the second frozen suspension to further drying conditions under reduced pressure to obtain a solid composition according to said first aspect.

According to a third aspect, the present invention is directed to a solid composition obtainable by the freeze-drying method according to the second aspect.

According to a fourth aspect, the present invention is directed to a glass vial containing a single dose of a Norovirus vaccine in solid form, such as a solid composition according to the first aspect of the present invention, obtainable by applying said single dose in form of an aqueous suspension to a wall of the vial cooled to a temperature of liquid nitrogen, allowing the suspension to freeze, and drying the suspension in the vial.

### BRIEF DESCRIPTION OF THE DRAWINGS

FIG. 1 Exemplarily plate-layout for the in vitro relative potency (IVRP) assay.

FIGS. 2A-2B Representative particle size distribution curves of Norovirus vaccine reconstituted from powders that were prepared by thin-film freeze-drying using various concentrations of trehalose (i.e., 0-5%, w/v) (2A) or various concentrations of sucrose (i.e., 0-5%, w/v) (2B). The measurements were repeated three times with similar results.

FIG. 3 Representative particle size distribution curves of bulk and single vial thin-film freeze dried Norovirus vaccine powders with exemplary formulations after reconstitution (i.e. 5% of sucrose or 4% of sucrose). The measurement was repeated with two or three vials, with similar results.

FIGS. 4A-4B The relative potency as measured by the IVRP assay for the strain GII.4 Consensus VLP antigen (4A). The relative potency as measured by the IVRP assay for the GI. 1 Norwalk VLP antigen (4B).

### Definitions

The term “VLPs” is defined for purposes of the present invention to refer to virus like particles. Virus like particles, such as Norovirus VLPs, are structurally similar to viruses, but lack the infectious genetic material and, therefore, are not infectious. VLPs are usually prepared by recombinant expression and spontaneous self-assembly of the capsid protein.

As used herein, the term “thin-film freeze-drying” (TFFD) is a specific freeze-drying method in which the liquid, e.g. a solution or a suspension, to be freeze-dried is dropped on a moving (e.g., rotating) cryogenic surface and frozen (typically at a freezing rate ranging from about 100 K/s to about 1000 K/s, or any ranges or subranges therebetween) in order to form a first frozen liquid in the form of a frozen film having a thickness of less than about 500 μm. In some embodiments, the thickness is less than about 50 μm, 60 μm, 70 μm, 80 μm, 90 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1 mm, including any values therebetween. “Moving” as used herein refers to the relative movement to each other of said cryogenic surface and a nozzle or other kind of orifice through which the liquid to be freeze dried is flowing, so that with reference to an external fixed point either the cryogenic surface, the orifice, or both can move, as long as the cryogenic surface and the orifice move in relation to each other. In some embodiments, moving includes rotating, oscillating, translating, tilting, separating, or any combination thereof. Said frozen film is collected in a container which is cooled to about the temperature of liquid nitrogen in order to form a second frozen liquid. This second frozen liquid is then subjected to further drying conditions similar to these of conventional shelf freeze-drying, such as a period of 20 to 100 hours and a temperature profile ranging from about −50° C. to about 30° C. at a pressure of less than about 500 mTorr. The terms “freeze-drying” and “lyophilization” are used interchangeably herein.

The term “solid composition” refers to a dry composition. In some embodiments, the term “dry” relates to a composition having a moisture content of less than about 5 wt.-%, more preferably of less than about 3 wt.-%, based on the total mass of the composition, wherein the moisture content is determined using a Karl Fischer titration method, such as the Karl Fischer titration method set forth in the Examples section. For example, a solid dry composition can be obtained by carrying out TFFD to an aqueous suspension. In some embodiments, a solid composition has a moisture content of less than about 5, 4, 3, 2, or 1 wt.-%, including any values therebetween.

“X₁₀, X₅₀, and X₉₀” denote values corresponding to 10%, 50% and 90% of the cumulative undersize distribution of the antigen particles. In other words, X₁₀, X₅₀ and X₉₀ each denote the dimension of an antigen particle at which 10%, 50% and 90%, respectively, of the antigen particles in the sample are smaller. Alternatively, X₁₀, X₅₀, and X₉₀ are also referred to as D₁₀, D₅₀ and D₉₀ in the art. X₁₀, X₅₀ and X₉₀ are determined using a laser diffraction method after an optional 2-fold dilution to reach an obscuration effect of ˜10%. In some embodiments, X₁₀, X₅₀ and X₉₀ may be determined by the laser diffraction method as described in the Examples section.

The term “aqueous suspension” describes a dispersion of a solid, such as antigen particles as described herein, the aqueous suspension having a water content of at least about 50 wt.-%, or at least about 60 wt.-% with respect to the total mass of the suspension, wherein less than 5 wt.-%, preferably less than 1 wt.-% of the dispersed solid, with respect to the total mass of the aqueous suspension, is dissolved. In some embodiments, the water content is at least about 50, 55, 60, 65, 70, 75, 80, 85, 90, or 95 wt.-%, including any values therebetween. In some embodiments, less than about 5, 4, 3, 2, or 1 wt.-% of the dispersed solid is dissolved with respect to the total mass of the aqueous solution. In some embodiments, the pH of the aqueous suspension is about 6.0, 6.1, 6.2, 6.3, 6.4, 6.5, 6.6, 6.7, 6.8, 6.9, or 7.0, or any ranges or subranges therebetween. In some embodiments, the pH of such aqueous suspension ranges from about 6 to about 7, preferably from about 6.4 to about 6.6.

An “in vitro relative potency” (IVRP) assay, e.g., an IVRP assay as described in the Examples section herein, is used to determine the relative potency of antigens. Therefore, the potency of antigens included in antigen particles in a reference aqueous suspension, e.g. a reference vaccine, which includes the same components in the same amounts as the solid composition and which has not been subjected to drying, and the potency of antigens included in antigen particles of a test vaccine (e.g. the solid composition mixed with water, such as distilled or sterile water, for reconstituting the solid composition to achieve an aqueous suspension of the antigen particles with a concentration of 100 μg/ml) which has been subjected to drying before reconstitution are determined. Afterwards, the relative potency is determined by dividing the potency of the test composition by the potency of the reference vaccine. In particular, a test composition is defined as “stable”, even under severe conditions, if the relative potency as determined by said IVRP assay is in an acceptance range ranging from about 50% to about 150%.

As used herein, “severe” storage conditions are storage conditions of the solid composition including an increased storage temperature of more than 8° C., such as room temperature (i.e. 20° C.) or 40° C., and relative humidity, such as 75%, for an extended period of time, such as 4 weeks. In some embodiments, severe storage conditions comprise a storage temperature of more than about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50° C., or any values therebetween. In some embodiments, severe storage conditions comprise storage in nonoptimal conditions for a period of at least about 2, 3, 4, 5, 6, 7, 8, 9, 10, 11, or 12 weeks. In some embodiments, severe storage conditions comprise exposure to humidity of at least about 60, 65, 70, 75, 80, 85, or 90%, or any values therebetween. In some embodiments, severe conditions relate to accelerated stability study conditions including a storage temperature of 40° C. and a relative humidity of 75% for an extended period of 4 weeks.

As used herein, a “reference vaccine”, used for determining the relative potency, is an aqueous suspension in which the same components (besides water) in the same absolute amounts as in the test vaccine (e.g., the solid composition mixed with water, such as distilled or sterile water, for reconstituting the solid composition to achieve an aqueous suspension of the antigen particles with a concentration of 100 μg/ml) are dispersed, i.e. the antigen particles described herein. In contrast to the test vaccine, the “reference aqueous suspension” or “reference vaccine” is not subjected to drying prior to measuring its potency. For determining the relative potency of the test vaccines as analyzed herein, the same reference vaccine is used in order to ensure comparability between different test vaccines. The reference vaccine is continuously stored at a temperature between 2° C. and 8° C. The “reference vaccine” and the test vaccine can be prepared independently from each other.

The term “antigen particles” refers to adjuvant particles on which antigens, including VLPs, are adsorbed and which are capable of inducing an antibody response.

The term “particle” as used herein refers to a solid which can be dispersed in an aqueous solution without essentially dissolving. In some embodiments, a particle has a dimension of at least about 0.1 μm, 0.2 μm, 0.3 μm, 0.4 μm, 0.5 μm, 0.6 μm, 0.7 μm, 0.8 μm, 0.9 μm, or 1.0 μm, or any values therebetween, when dispersed in an aqueous solution. In some embodiments, a particle has a dimension of at least about 0.5 μm when dispersed in an aqueous solution.

The term “species” in accordance with the International Committee on Taxonomy of Viruses is defined as a monophyletic group of viruses whose properties can be distinguished from those of other species by multiple criteria. Norovirus is an example of a virus species.

The term “genotype” is defined for purposes of the present invention to refer to the genetic constitution of an organism, such as the Norovirus genotypes within genogroups GI, GII, GIII and GIV.

As used herein, “immunogenic composition” refers to a composition comprising antigen particles and which is capable of eliciting an immune response in a subject by virtue of its antigen particles. Within that context the subject may be an animal or a human.

The term “vaccine” or “vaccine composition” is defined for purposes of the present invention to refer to a formulation which contains VLPs as disclosed herein in a form that is capable of being administered to a mammal and which elicits protective immunity to a viral infection in a mammal.

The term “adjuvant particle” is defined for purposes of the present invention to refer to a compound added to the aqueous composition in order to lead to an enhanced immune response, for instance when used as a mammalian vaccine. The adjuvant particle may for example be an aluminum salt.

The term “Norovirus” is defined for purposes of the present invention to refer to members of the species Norovirus of the family Caliciviridae. In some embodiments, a Norovirus can include a group of related, positive-sense single-stranded RNA, non-enveloped viruses that can be infectious to human or non-human mammalian species. In some embodiments, Noroviruses can cause acute gastroenteritis in humans. Included within the group of Noroviruses are at least four genotypes (GI, GII, GIII, GIV) defined by nucleic acid and amino acid sequences which comprise 15 genetic clusters. The major genotypes are GI and GII. Non-limiting examples of Noroviruses include Norwalk virus (NV, GenBank M87661, VP1 sequence NP_056821), Southampton virus (SHV, GenBank L07418), Desert Shield virus (DSV, GenBank U04469), Hesse virus (HSV), Chiba virus (CHV, GenBank AB042808), Hawaii virus (HV, GenBank U07611), Snow Mountain virus (SMV, GenBank U70059), Toronto virus (TV, Leite et al., Arch. Virol. 141:865-875), Bristol virus (BV), Jena virus (JV, GenBank AJ011099), Maryland virus (MV, GenBank AY032605), Seto (Aichi) virus (SV, GenBank AB031013), Camberwell (CV, GenBank AF145896), Lordsdale virus (LV, GenBank X₈₆₅₅₇), Grimsby virus (GrV, GenBank AJ004864), Mexico virus (MXV, GenBank U22498), Boxer (GenBank AF538679), C59 (GenBank AF435807), VA115 (GenBank AY038598), BUDS (GenBank AY660568), Houston virus (HoV, GenBank EU310927), MOH (GenBank AF397156), Paris Island (PiV, GenBank AY652979), VA387 (GenBank AY038600), VA207 (GenBank AY038599), and Operation Iraqi Freedom (OIF, GenBank AY675554). Also included are Norovirus consensus VLPs, which are a construct representing a consensus sequence from two or more Norovirus strains such as GII.4 strains. Also included are Norovirus composite VLPs, which are derived from a consensus sequence from two or more Norovirus strains such that the consensus sequence contains at least one different amino acid as compared to each of the sequences of said two or more Norovirus strains. The construction of Norovirus consensus VLPs is disclosed in WO 2010/017542, which is herewith incorporated by reference in its entirety.

### DETAILED DESCRIPTION

### VLP Species and Production

Virus-like particle(s) or VLP(s) refer to virus-like particle(s), produced from the capsid protein-coding sequence of a virus, particularly a non-enveloped virus, and comprising antigenic characteristic(s) similar to those of the infectious virus.

In some embodiments, the VLP is produced from the capsid protein of a non-enveloped virus. In some embodiments, the non-enveloped virus is selected from the group consisting of Calicivirus (e.g., a Norovirus or a Sapovirus), Picornavirus, Astrovirus, Adenovirus, Reovirus, Polyomavirus, Papillomavirus, Parvovirus, and Hepatitis E virus.

In some embodiments, the VLPs are derived from at least 1 species of virus, or from at least 2 different species of virus, or from at least 3 different species of virus, or from at least 4 different species of virus.

In some embodiments, the VLPs are selected from the group of species of Norovirus VLPs, Rotavirus VLPs, HPV VLPs, Influenza virus VLPs, Corona virus VLPs, and hepatitis B virus VLPs. In some preferred embodiments, the aqueous composition may comprise VLPs selected from the group of species of Norovirus VLPs and Rotavirus VLPs.

In some embodiments, the VLPs comprise at least 1 genotype, or the VLPs comprise at least 2 different genotypes, or the VLPs comprise at least 3 different genotypes, or the VLPs comprise at least 4 different genotypes. In some embodiments, the VLPs comprise a mixture of VLPs that are individually composed of capsid proteins from individual genotypes. For example, in some embodiments, the VLPs comprise Norovirus genotype GI.1 VLPs and Norovirus genotype GII.4 VLPs. In some embodiments, the VLPs are composed of consensus capsid protein sequences representing multiple genotypes.

In some embodiments, the VLPs comprise one or more different genotypes of Norovirus VLP selected from the group consisting of Norovirus genogroup I (GI) VLPs, Norovirus genogroup II (GII) VLPs, Norovirus genogroup III (GIII) VLPs, and Norovirus genogroup IV (GIV) VLPs. In some preferred embodiments the genotypes of the Norovirus VLPs may be selected from the group of Norovirus genogroup I (GI) VLPs and Norovirus genogroup II (GII) VLPs. In some more preferred embodiments, the genotypes of the Norovirus VLPs may be selected from the group of Norovirus genotype I.1 (GI.1) VLPs, Norovirus genotype II.2 (GII.2) VLPs and Norovirus genotype II.4 (GII.4) VLPs, for example, Norwalk GI.1 virus VLPs and a construct representing a consensus sequence or a composite sequence from several norovirus GII.4 strains. Furthermore, it is preferred that VLPs consists of a Norovirus genotype GI.1 Norwalk VLP and Norovirus genotype GII.4 Consensus VLP. For example, in some embodiments, the consensus sequence or composite sequence is derived from Norovirus strains selected from the group consisting of Houston strain, Minerva strain and Laurens strain. In some embodiments, the consensus sequence of the Norovirus strain GII.4 Consensus VLP is represented by SEQ ID NO: 1. In some embodiments, the sequence of the Norovirus genotype GI.1 Norwalk VLP is represented by SEQ ID NO: 2. In some embodiments, the sequence of the Norovirus genotype GI.1 Norwalk VLP is represented by SEQ ID NO: 3. In some embodiments, the consensus sequence of the Norovirus genotype GII.4 Consensus VLP is represented by SEQ ID NO: 1 and the sequence of the Norovirus genotype GI.1 Norwalk VLP is represented by SEQ ID NO: 2 or 3. In some embodiments, the sequences have at least 70%, 75%, 80%, 85%, 90%, 95%, or 99% identity to SEQ ID NO: 1, 2, or 3. Norovirus VLPs may be produced by methods disclosed in WO 2010/017542 which is incorporated by reference herein in its entirety. The contents of each of the following patent publications are also incorporated herein by reference in their entireties and for all purposes: WO 2008/042789; WO 2008/113011; WO 2009/039229; WO 2010/017542; WO 2013/009849; WO 2013/192604; WO 2020/041192; WO 2020/132510; and WO 2015/051255.

Sequences of VLPs of interest are provided in Table 1 below.

TABLE 1
Exemplary Norovirus VLP sequences

| SEQ | |
|---|---|
| ID | |
| NO | Sequence |
| 1 | MKMASSDANPSDGSTANLVP |
| | EVNNEVMALEPVVGAAIAAP |
| | VAGQQNVIDPWIRNNFVQAP |
| | GGEFTVSPRNAPGEILWSAP |
| | LGPDLNPYLSHLARMYNGYA |
| | GGFEVQVILAGNAFTAGKII |
| | FAAVPPNFPTEGLSPSQVTM |
| | FPHIIVDVRQLEPVLIPLPD |
| | VRNNFYHYNQSNDPTIKLIA |
| | MLYTPLRANNAGDDVFTVSC |
| | RVLTRPSPDFDFIFLVPPTV |
| | ESRTKPFTVPILTVEEMTNS |
| | RFPIPLEKLFTGPSGAFVVQ |
| | PQNGRCTTDGVLLGTTQLSP |
| | VNICTFRGDVTHIAGTQEYT |
| | MNLASQNWNNYDPTEEIPAP |
| | LGTPDFVGKIQGVLTQTTRG |
| | DGSTRGHKATVSTGSVHFTP |
| | KLGSVQFSTDTSNDFETGQN |
| | TKFTPVGVVQDGSTTHQNEP |
| | QQWVLPDYSGRDSHNVHLAP |
| | AVAPTFPGEQLLFFRSTMPG |
| | CSGYPNMNLDCLLPQEWVQH |
| | FYQEAAPAQSDVALLRFVNP |
| | DTGRVLFECKLHKSGYVTVA |
| | HTGQHDLVIPPNGYFRFDSW |
| | VNQFYTLAPMGNGTGRRRAL |
| 2 | MMMASKDATSSVDGASGAGQ |
| | LVPEVNASDPLAMDPVAGSS |
| | TAVATAGQVNPIDPWIINNF |
| | VQAPQGEFTISPNNTPGDVL |
| | FDLSLGPHLNPFLLHLSQMY |
| | NGWVGNMRVRIMLAGNAFTA |
| | GKIIVSCIPPGFGSHNLTIA |
| | QATLFPHVIADVRTLDPIEV |
| | PLEDVRNVLFHNNDRNQQTM |
| | RLVCMLYTPLRTGGGTGDSF |
| | VVAGRVMTCPSPDFNFLFLV |
| | PPTVEQKTRPFTLPNLPLSS |
| | LSNSRAPLPISSIGISPDNV |
| | QSVQFQNGRCTLDGRLVGTT |
| | PVSLSHVAKIRGTSNGTVIN |
| | LTELDGTPFHPFEGPAPIGF |
| | PDLGGCDWHINMTQFGHSSQ |
| | TQYDVDTTPDTFVPHLGSIQ |
| | ANGIGSGNYVGVLSWISPPS |
| | HPSGSQVDLWKIPNYGSSIT |
| | EATHLAPSVYPPGFGEVLVF |
| | FMSKMPGPGAYNLPCLLPQE |
| | YISHLASEQAPTVGEAALLH |
| | YVDPDTGRNLGEFKAYPDGF |
| | LTCVPNGASSGPQQLPINGV |
| | FVFVSWVSRFYQLKPVGTAS |
| | SARGRLGLRR |
| 3 | MMMASKDATSSVDGASGAGQ |
| | LVPEVNASDPLAMDPVAGSS |
| | TAVATAGQVNPIDPWIINNF |
| | VQAPQGEFTISPNNTPGDVL |
| | FDLSLGPHLNPFLLHLSQMY |
| | NGWVGNMRVRIMLAGNAFTA |
| | GKIIVSCIPPGFGSHNLTIA |
| | QATLFPHVIADVRTLDPIEV |
| | PLEDVRNVLFHNNDRNQQTM |
| | RLVCMLYTPLRTGGGTGDSF |
| | VVAGRVMTCPSPDFNFLFLV |
| | PPTVEQKTRPFTLPNLPLSS |
| | LSNSRAPLPISSMGISPDNV |
| | QSVQFQNGRCTLDGRLVGTT |
| | PVSLSHVAKIRGTSNGTVIN |
| | LTELDGTPFHPFEGPAPIGF |
| | PDLGGCDWHINMTQFGHSSQ |
| | TQYDVDTTPDTFVPHLGSIQ |
| | ANGIGSGNYVGVLSWISPPS |
| | HPSGSQVDLWKIPNYGSSIT |
| | EATHLAPSVYPPGFGEVLVF |
| | FMSKMPGPGAYNLPCLLPQE |
| | YISHLASEQAPTVGEAALLH |
| | YVDPDTGRNLGEFKAYPDGF |
| | LTCVPNGASSGPQQLPINGV |
| | FVFVSWVSRFYQLKPVGTAS |
| | SARGRLGLRR |

### Adjuvant Particles

Various methods of achieving an adjuvant effect for vaccines are known and may be used in conjunction with the VLPs disclosed herein. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, the contents of which are incorporated by reference herein.

In some embodiments, exemplary adjuvant particles include, but are not limited to, aluminum salts, calcium phosphatechitosan, poly(lactide-co-glycolides) (PLG) microparticles, poloxamer particles, and microparticles. In some embodiments, the adjuvant particle is an aluminum salt.

In some embodiments, the adjuvant includes at least one of an aluminum salt (such as aluminum hydroxide), aluminum phosphate, aluminum (oxy)hydroxide, aluminum hydroxide, precipitated aluminum hydroxide, potassium aluminum sulfate, and gel-like aluminum hydroxide such as, e.g. Alhydrogel 85. Hereinafter, aluminum oxide hydroxide, aluminum hydroxide and precipitated and/or gel-like aluminum hydroxide in a pharmaceutically acceptable form, in particular for use as adjuvants, are also collectively referred to as “aluminum hydroxide”.

In some embodiments, the adjuvant is aluminum hydroxide.

### Antigen Particles

Antigen particles according to the present invention comprise antigen particles comprising virus like particles (VLPs), as described above, adsorbed on adjuvant particles, as described above. Such antigen may be obtainable by aseptically blending VLPs and adjuvant particles in a solvent, e.g. an aqueous solvent.

Without wishing to be bound by any theory, adsorbing one or more VLP antigen(s) (e.g., a Norovirus VLP) to an aluminum salt (such as e.g. aluminum hydroxide) may enhance the stability of the VLP antigen.

In some embodiments, the antigen particles have a cumulative undersize distribution with an X₉₀ value of less than about 30 μm, as determined by laser diffraction. In some embodiments, the X₉₀ value is less than about 40, 35, 30, 25, 20, 15, or 10 μm, or any values therebetween. In some embodiments, the X₉₀ value is less than about 20 μm or less than about 15 μm. In some embodiments, the X₉₀ values are measured after reconstitution of a solid composition including these antigen particles, wherein said solid composition results from a freshly prepared aqueous suspension which has been e.g. subjected to freeze-drying, such as TFFD. In some embodiments, the X₉₀ values are comparable with the X₉₀ values of a reference aqueous suspension, as defined herein, including the same antigen particles in the same amount. Thus, in some embodiments, such X₉₀ values are an indication that the particles of the solid composition did not significantly agglomerate upon freeze-drying. Without wishing to be bound by any theory, it is assumed that such prevention of the agglomeration also preserves the potency of the VLP antigens in the antigen particles. For example, prevention of agglomeration may preserve the physical characteristics of the VLP antigens, the dispersion of the VLP antigens, the exposed surface area of the VLP antigens, and the like.

In some embodiments, the antigen particles have a cumulative undersize distribution with an X₉₀ value of more than about 5 μm to less than about 30 μm, as determined by laser diffraction. In some embodiments, the X₉₀ value is in the range of about 1 μm to 30 μm, about 5 μm to 30 μm, about 5 μm to 20 μm, or about 5 μm to 15 μm, or any ranges or subranges therebetween. In some embodiments, the X₉₀ value is more than about 5 μm to less than about 20 μm. In some embodiments, the X₉₀ value is more than about 5 μm to less than about 15 μm.

In some embodiments, in addition or alternatively to the X₉₀ values disclosed above, the antigen particles have an X₅₀ value, as determined by laser diffraction. In some embodiments, the X₅₀ value is less than about 10, 9, 8, 7, 6, 5, 4, 3, or 2 μm, or any values therebetween. In some embodiments, the antigen particles have an X₅₀ value of less than about 5 μm. In some embodiments, the antigen particles have an X₅₀ value of less than about 4.5 μm. In some embodiments, the X₅₀ value is in the range of 0.5-20 μm, 1-15 μm, 1-10 μm, 2-5 μm, 2.1-4.5 μm, or any ranges or subranges therebetween. In some embodiments, said X₅₀ value ranges from more than about 2 μm to less than about 5 μm or from more than about 2.1 μm to less than about 4.5 μm.

In some embodiments, in addition or alternatively to the X₉₀ values and/or the X₅₀ values disclosed above, the antigen particles have an X₁₀ value, as determined by laser diffraction. In some embodiments, the X₁₀ value is less than about 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1 μm, or any values therebetween. In some embodiments, the X₁₀ value is less than about 2 μm or less than about 1.9 μm. In some embodiments, said X₁₀ value ranges from more than about 0.7 μm to less than about 2 μm or from more than about 0.8 μm to less than about 1.9 μm.

### Solid Composition

According to a first aspect, the present invention is directed to a solid composition comprising: antigen particles, as described above, comprising virus like particles (VLPs), as described above, adsorbed on adjuvant particles as described above. In some embodiments, said solid composition is obtained by drying an aqueous suspension, such as a freshly prepared aqueous suspension, including the same components in the same amounts. In some embodiments, said solid composition is obtained by freeze-drying an aqueous suspension, such as a freshly prepared aqueous suspension, including the same components in the same amounts. In some embodiments, said solid composition is obtained by carrying out TFFD on an aqueous suspension, such as a freshly prepared aqueous suspension, including the same components in the same absolute amounts as in the freshly prepared aqueous suspension.

In some embodiments, the antigen particles have a relative potency of at least about 50%, wherein said relative potency is determined by measuring the potency of the antigens in the antigen particles of a reference aqueous suspension of the composition, as defined herein, which has not been subjected to drying and the potency of the antigens in the antigen particles of the solid composition, based on the potency of said reference aqueous suspension. In some embodiments, the relative potency is at least about 50, 60, 70, 80, 90, 100, 110, 120, 130, 140, 150, 160, 170, 180, 190, or 200%, or any value therebetween. In some embodiments, the relative potency is in the range of 50-100%, 75-150%, or 50-150%, or any ranges or subranges therebetween. In some embodiments, said relative potency ranges from about 50% to about 150%, including all ranges and subranges therebetween.

In some embodiments, the antigen particles have a relative potency of at least about 50, 60, 70, 80, 90, 100, 110, 120, 130, 140, or 150%, or any value therebetween after storage in severe storage conditions. In some embodiments, the relative potency is in the range of 50-100%, 75-150%, or 50-150%, or any ranges or subranges therebetween. In some embodiments, the severe storage conditions comprise storage at a temperature of at least about 10, 15, 20, 25, 30, 35, 40, 45, 50, 55, or 60° C., or any ranges or subranges therebetween. In some embodiments, the antigen particles are stored at a relative humidity of at least about 50, 55, 60, 65, 70, 75, 80, 85, or 90%. In some embodiments, the antigen particles are stored in severe storage conditions for at least 1, 2, 3, 4, 5, 6, or 7 days or at least 1, 2, 3, 4, 5, 6, 7, 8, 9, 10, 11, or 12 weeks, or any values therebetween. In some embodiments, the antigen particles continue to have a relative potency of at least about 50% after storage at about 40° C. and about 75% relative humidity (RH) for at least 4 weeks. In some embodiments, the antigen particles continue to have a relative potency ranging from about 50% to about 150% after storage at about 40° C. and about 75% relative humidity (RH) for at least 4 weeks. In some embodiments, the potency of the antigen particles of the solid composition does not significantly change or does not significantly decrease in an accelerated stability study. This indicates that using the solid composition is a viable approach in addressing the cold-chain requirement, i.e. that a storage of the antigen particles under a temperature ranging from 2° C. to 8° C. is not needed.

In some embodiments, the solid composition is a dry composition having a moisture content of less than about 10, 9, 8, 7, 6, 5, 4, 3, 2, or 1 wt.-% based on the total mass of the dry composition, wherein said moisture content is determined using a Karl Fischer titration method. In some embodiments, it has a moisture content of less than about 5 wt.-%, less than about 3 wt.-%, or less than about 2 wt.-%. In some embodiments, low moisture content is achieved by carrying out TFFD on an aqueous suspension of the composition, such as a freshly prepared aqueous suspension of the composition.

In some embodiments, the solid composition further comprises a sugar or a sugar alcohol. In some embodiments, the sugar is a monosaccharide, a disaccharide, an oligosaccharide, or a polysaccharide. In some embodiments, the sugar is glucose, fructose, galactose, sucrose, lactose, maltose, or trehalose. In some embodiments, the sugar is trehalose, lactose, or sucrose. In some embodiments, the sugar is sucrose. In some embodiments, the sugar alcohol is ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, or polyglycitol. In some embodiments, the sugar alcohol is mannitol.

In some embodiments, the mass ratio between the antigen particles and the sugar or sugar alcohol, based on the mass of the sugar or sugar alcohol in the solid composition, is in the range of 2×10⁻² to 7×10⁻², including any ranges or subranges therebetween. In some embodiments, a mass ratio between the antigen particles and the sugar or sugar alcohol, based on the mass of the sugar or sugar alcohol in the solid composition, is less than about 7×10⁻², 6×10⁻², 5×10⁻², 4×10⁻², 3×10⁻², or 2×10⁻². In some embodiments, a mass ratio between the antigen particles and the sugar or sugar alcohol, based on the mass of the sugar or sugar alcohol in the solid composition, is less than about 4.2×10⁻². In some embodiments, the dry content of the sugar or sugar alcohol is at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 wt.-%, or any ranges or subranges therebetween, wherein the dry content is calculated based on the moisture-free solid composition. In some embodiments, the dry content of the sugar or sugar alcohol is at least about 60 wt.-%. In some embodiments, the mass ratio between the antigen particles and the sugar, such as trehalose combined with sucrose, is at least about 2.68×10⁻². In some embodiments, the mass ratio between the antigen particles and sucrose is about 2.8×10⁻² or less. In some embodiments, the mass ratio ranges from about 2.8×10⁻² to about 2.0×10⁻². In some embodiments, the dry combined contents of all sugars and sugar alcohols is at least about 60 wt.-%, 65 wt.-%, 70 wt.-%, or 75 wt.-%,. In some embodiments, the dry combined contents of trehalose and sucrose is about 75.75 wt.-%, or the dry content of sucrose is at least about 75.75 wt.-%. It has been surprisingly found that, if the mass ratio of antigen particles to sugar and/or the dry content of the sugar are in the disclosed ranges or encompasses a particular value of these ranges, the particle agglomeration can be further prevented. In some embodiments, the weight percent of sugar and/or sugar alcohol in the aqueous solution is 1-5%, 2-5%, or 2-4%, or any ranges or subranges therebetween, wherein the aqueous solution comprises the solid components of the reference vaccine at a concentration comparable to the concentration within the reference vaccine or at least about 80% to about 110% of the concentration of the reference vaccine.

In some embodiments, a total mass of the solid composition, calculated based on its mosture-free form (i.e. a moisture content of 0.0 wt.-% based on the total mass of the solid composition), may range from about 10 mg to about 60 mg, or from about 15 mg to about 50 mg. In some embodiments, the total mass may range from about 30 mg to about 50 mg. More preferably, the total mass of the solid composition may be about 15.69 mg, about 21.8 mg, about 26.8 mg, 31.8 mg, 36.8 mg, 41.8 mg or 46.8 mg.

In some embodiments, the solid composition comprises from about 10 μg to about 100 μg of Norovirus strain GI.1 Norwalk VLPs, from about 30 μg to about 200 μg of Norovirus strain GII.4 Consensus VLPs and from about 300 μg to about 700 μg of adjuvant. In some embodiments, the composition comprises from 400 μg to about 600 μg or about 500 μg of adjuvant. In some embodiments, said composition comprises at least about 15, 16, 17, 18, 19, 20, 21, 22, 23, 24, 25, 26, 27, 28, 29, or 30 mg of a sugar or sugar alcohol, or any ranges or subranges therebetween. In some embodiments, said composition comprises at least about 20 mg of a sugar or sugar alcohol. In some embodiments, the solid composition comprises about 20 mg of trehalose and optionally about 6.11 mg of sucrose. In some embodiments, the solid composition comprises at least about 20 mg of sucrose to at most about 25 mg or about 30 mg of sucrose, or any ranges or subranges therebetween.

In some embodiments, the VLPs are Norovirus strain GI.1 Norwalk VLPs and Norovirus strain GII.4 Consensus VLPs, wherein the composition comprises from about 15 μg to about 50 μg of Norovirus strain GI.1 Norwalk VLP from about 50 μg to about 150 μg of Norovirus strain GII.4 Consensus VLPs and about 500 μg of adjuvant, wherein the adjuvant is aluminum hydroxide in the form of Al(OH)3. In a preferred embodiment, the VLPs are Norovirus strain GI.1 Norwalk VLPs and Norovirus strain GII.4 Consensus VLPs, wherein the composition comprises about 15 μg of Norovirus strain GI.1 Norwalk VLPs, about 50 μg of Norovirus strain GII.4 Consensus VLPs and about 500 μg of adjuvant, wherein the adjuvant is aluminum hydroxide in the form of Al(OH)3. In an alternatively preferred embodiment, the VLPs are Norovirus strain GI.1 Norwalk VLPs and Norovirus strain GII.4 Consensus VLPs, wherein the composition comprises about 50 μg of Norovirus strain GI.1 Norwalk VLPs, about 150 μg of Norovirus strain GII.4 Consensus VLPs and about 500 μg of adjuvant, wherein the adjuvant is aluminum hydroxide in the form of Al(OH)3. In a more preferred embodiment, the composition additionally comprises about 20 mg, 25 mg, about 26.11 mg, or about 31.11 mg of sucrose. In another more preferred embodiment, the composition additionally comprises about 20 mg of trehalose and, optionally, about 6.11 mg of sucrose.

In some embodiments, the solid composition is an immunogenic composition. In some embodiments, said immunogenic composition is a vaccine composition.

In some embodiments, the vaccine composition has a pH ranging from about 6 to about 7. In some embodiments, the vaccine composition has a pH of about 6.0, 6.1, 6.2, 6.3, 6.4, 6.5, 6.6, 6.7, 6.8, 6.9, or 7.0, or any values therebetween. In some embodiments, the vaccine has a pH ranging from about 6.4 to about 6.6 upon mixing with about 0.5 ml of water or a buffer solution.

In some embodiments, the vaccine composition has an Osmolality ranging from about 200 to about 600 upon mixing with about 0.5 ml water or a buffer solution. In some embodiments, the vaccine composition has an osmolality of at least about 200, 250, 300, 350, 400, 450, 500, 550, or 600, or any values therebetween, upon mixing with about 0.5 ml water or a buffer solution.

In some embodiments, the vaccine composition includes additional vaccine components selected from the group consisting of a salt, a buffer, such as e.g. an amino acid, a surfactant and an inorganic acid. In some embodiments, the buffer includes phosphate, e.g., potassium phosphate, acetate and/or Tris. In some embodiments, the salt is sodium chloride. In some embodiments, the buffer is the amino acid L-histidine, glycine, lysine, albumins (HSA and/or BSA) and/or proline. In some embodiments, the inorganic acid is hydrochloric acid. In some embodiments, the additional vaccine components consist of sodium chloride, L-histidine and hydrochloric acid. In some embodiments, the surfactant is Tween-80, Poloxamer, polyvinyl alcohol (PVA) and/or other stabilizers known to the person skilled in the art. In some embodiments, the composition comprises from about 3 to about 5 mg of the salt, from about 0.5 to about 3 mg of an amino acid as the buffer and from about 0.5 to about 2.5 mg of the inorganic acid, including any ranges or subranges within these ranges.

In some embodiments, the solid composition is a vaccine composition and the VLPs are Norovirus genotype GI.1 NorwalkVLPs and Norovirus genotype GII.4 ConsensusVLPs, wherein the composition comprises about 15 μg of Norovirus genotype GI.1 NorwalkVLPs, about 50 μg of Norovirus genotype GII.4 ConsensusVLPs and about 500 μg of adjuvant, wherein the adjuvant is aluminum hydroxide in the form of Al(OH)₃, and wherein the vaccine composition includes further vaccine components consisting of 4.38 mg sodium chloride, 1.55 mg L-histidineand 1.73 mg HCl.

In some embodiments, the solid composition is a vaccine composition and the VLPs are Norovirus genotype GI.1 NorwalkVLPs and Norovirus genotype GII.4 ConsensusVLPs, wherein the composition comprises about 50 μg of Norovirus genotype GI.1 NorwalkVLPs, about 150 μg of Norovirus genotype GII.4 ConsensusVLPs, and about 500 μg of adjuvant, wherein the adjuvant is aluminum hydroxide in the form of Al(OH)₃, and wherein the vaccine composition includes further vaccine components comprising 4.38 mg sodium chloride, 1.55 mg L-histidine and 1.73 mg HCl. In some embodiments, the vaccine composition additionally comprises 6.11 mg of sucrose and 20 mg of trehalose. In an alternative preferred embodiment, the composition comprises at least about 20 mg or about 26.11 mg of sucrose and at most about 30 mg or 36.11 mg of sucrose.

### Freeze-Drying Method

According to a second aspect, the present invention is directed to a freeze-drying method for removing water from an aqueous suspension, such as a freshly prepared aqueous suspension, the aqueous suspension comprising:
antigen particles, as described herein, comprising virus like particles, as described herein, adsorbed on adjuvant particles as described herein;
the method comprising the steps:
step 1: providing the aqueous suspension optionally at a temperature ranging from about 2° C. to about 30° C.;
step 2: decreasing the temperature of step 1 with a freezing rate of at least 50 K/s to obtain a first frozen suspension;
step 3: collecting the frozen suspension in a container cooled to the temperature of liquid nitrogen to obtain a second frozen suspension;
step 4: subjecting the second frozen suspension to further drying conditions under reduced pressure to obtain a solid composition according to the second aspect of the present invention.

In some embodiments, the temperature at which the aqueous suspension is provided in step 1 ranges from about 2° C. to about 30° C., or from 2° C. to about 27° C., or from 2° C. to about 25° C. In a some embodiments, the temperature ranges from about 2° C. to about 8° C.

In some embodiments, the aqueous suspension is prepared at a temperature ranging from about 20° C. to about 30° C., in particular at a temperature ranging from about 22° C. to about 27° C. and, more preferably, at a temperature at about 25° C. before the aqueous suspension is provided in step 1.

In certain embodiments, the first frozen suspension in step 2 is formed as a film having a thickness of less than about 500 μm or less than about 450 μm. Preferably, the thickness of the film ranges from about 50 μm to about 500 μm or from about 80 to about 450 μm. In a preferred embodiment, said film is formed by contacting the aqueous suspension with a moving cryogenic surface. Such moving cryogenic surface may be a surface of a rotating ring body, such as a cylinder, e.g. a drum, or a plate. In a preferred embodiment, the diameter of the rotating body ranges from about 2 cm to about 20 cm or from about 5 to about 10 cm. More preferably, the diameter of the rotating body is about 10 cm. Furthermore, a moving cryogenic surface may be a surface of a conveyor belt. In case of the surface of a rotating ring body, such as a cylinder or a drum, the rotating speed of the moving surface may range from about 2 rpm to about 20 rpm or from about 3 rpm to about 10 rpm, preferably from about 5 rpm to about 7 rpm. Such rotating speed may be in particular be used in order to avoid the overlap of droplet. In a preferred embodiment, the track velocity of the rotating ring body, such as a cylinder or a drum, ranges from about 1.05×10−2 m/s to about 1.05×10−1 m/s or from about 1.57×10-2 to about 5.24×10-2, preferably from about 2.62×10−2 m/s to 3.67×10−2 m/s.

In some embodiments, the freezing rate in step 1 ranges from about 50 K/s to about 1500 K/s. In some embodiments, the freezing rate is at least about 50 K/s, 100 K/s, 150 K/s, 200 K/s, 250 K/s, 300 K/s, 350 K/s, 400 K/s, 450 K/s, 500 K/s, 550 K/s, 600 K/s, 650 K/s, 700 K/s, 750 K/s, 800 K/s, 850 K/s, 900 K/s, 950 K/s, 1000 K/s, 1100 K/s, 1200 K/s, 1300 K/s, 1400 K/s, or 1500 K/s, or any values therebetween. In some embodiments, the freezing rate in step 1 ranges from about 50 k/s to about 1100 K/s or from about 70 K/s to about 500 K/s or from about 80 K/s to about 200 K/s. For example, in some embodiments, the freezing rate in step 1 is about 100 K/s. Said freezing rate can e.g. be calculated based on the method disclosed in J. D. Engstrom et al., Pharmaceutical Research 2008; 25(6): 1334-1346. It has been surprisingly found that a freezing rate within the above-defined ranges contributes to the suppression of particle aggregation upon freezing. Without wishing to be bound by any theory, it is assumed that, if the freezing rate is too slow, which is e.g. the case when freezing in conventional lyophilization processes, large ice crystals form and disrupt the antigen particles' hydration shell by depleting water molecules, resulting in antigen unfolding and antigen particle aggregation. In contrast, the rapid freezing rate of the process according to the present invention helps to create numerous, small nucleated ice crystals rapidly, leaving very thin channels between the frozen ice crystals. The thin channels limit the chance of collisions between antigen particles and thus reduce their aggregation.

In some embodiments, the further drying conditions in step 4 include freeze-drying of the second frozen suspension over a period of time within a temperature range and at reduced pressure. In some embodiments, the period of time is between 10-100 hours, 10-50 hours, 20-40 hours, or any ranges or subranges therebetween. In some embodiments, the temperature range is −50° C. to 30° C., or any ranges or subranges therebetween. In some embodiments, the reduced pressure is less than atmospheric pressure. In some embodiments, the pressure is less than about 700, 650, 600, 550, 500, 450, 400, 350, 300, 250, 200, 150, 100, or 50 mTorr, or any values therebetween. In some embodiments, the further drying conditions comprise freeze-drying the frozen suspension over a period of 20 to 100 hours and a temperature profile ranging from about −50° C. to about 30° C. at a pressure of less than about 500 mTorr, in particular less than 200 mTorr. In some embodiments, a loading temperature in step 4 may range from about −50° C. to about −20° C. In addition, an initial (or “primary”) drying temperature in step 4 may range from about −50° C. to about −20° C. A primary drying time in step 4 may range from about 10 hours to about 30 hours and may in particular be 20 hours. A ramp to subsequent (or “secondary”) drying in step 4 may have a duration ranging from about 10 hours to about 30 hours. A temperature for secondary drying in step 4 may range from about 15° C. to about 30° C. and may in particular be 25° C. A duration for secondary drying in step 4 may range from about 10 hours to about 30 hours and may in particular be 20 hours.

In some embodiments, the antigen particles have a relative potency of at least about 50% or a relative potency ranging from at least 50% to at least 150%, determined by measuring the potency of the antigen particles before and after the composition is dried and based on the potency before the composition is dried using an in vitro relative potency (IVRP) assay. In some embodiments, the antigen particles have a relative potency as described herein. In some embodiments, the antigen particles have a relative potency following storage in severe storage conditions, as describe herein.

In some embodiments, the freeze-drying process according to the second aspect of the present invention is a TFFD. In some embodiments, the aqueous suspension such as a freshly prepared aqueous suspension to be freeze-dried is dropped onto a moving (e.g., rotating) cryogenic surface and frozen at a freezing rate ranging from about 100 K/s to about 1100 K/s in order to form a first frozen liquid in the form of a frozen film having a thickness of less than about 500 μm. This frozen film is collected in a container which is cooled to a temperature of liquid nitrogen in order to form a second frozen liquid. This second frozen liquid is then subjected to further drying conditions similar to these of conventional shelf freeze-drying, such as a period of 20 to 100 hours and a temperature profile ranging from about −50° C. to about 30° C. at a pressure of less than about 500 mTorr.

In some embodiments, the aqueous suspension has a sugar or sugar alcohol content of at least about 2% (w/v) or at least about 2.4% (w/v) with respect to the total volume of said suspension. In some embodiments, the aqueous suspension has a content of trehalose of at least about 4% (w/v) and, optionally at least about 0.4% (w/v) of sucrose, or a content of sucrose of at least about 4% (w/v) or at least about 4.4% (w/v).

Additional features relevant to the disclosure may be adapted from vacuum-foam drying (Pisal S et al., AAPS Pharm Sci Tech. 2006; 7:60), spray drying (see Chen D et al., Vaccine. 2010; 28:5093-5099.), spray freeze-drying (Maa Y F et al., J. Pharm. Sci. 2003; 92:319-332.), spray freezing into liquid (see Yu Z et al., Eur. J. Pharm. Sci. 2006; 27:9-18.), and spray freeze-drying of vaccines with aluminum salts (see Chen D and Kristensen D, Rev. Vaccines. 2009; 8:547-557.; and Overhoff K A et al., J Drug Del. Sci. Tech. 2009; 19:89-98). The contents of each of the foregoing publications are incorporated by reference herein.

In one aspect, the disclosure provides a freeze-drying method for removing water from an aqueous suspension of antigen particles comprising virus like particles (VLPs) adsorbed on adjuvant particles, wherein the VLPs are Norovirus VLPs, the method comprising the steps of: providing the aqueous suspension optionally at a temperature ranging from about 2° C. to about 30° C.; decreasing the temperature of step (a) with a freezing rate of at least 50 K/s to obtain a first frozen suspension; collecting said first frozen suspension in a container cooled to the temperature of liquid nitrogen to obtain a second frozen suspension; subjecting said second frozen suspension to further drying conditions under reduced pressure to obtain a solid composition according to any one of the embodiments disclosed herein. In some embodiments, the composition comprises one or more different genogroups of Norovirus VLPs selected from the group consisting of Norovirus genogroup 1 (GI) VLPs and Norovirus genogroup 2 (GII) VLPs. In some embodiments, the VLPs consist of Norovirus genotype GI. 1 Norwalk VLPs and Norovirus genotype GII.4 ConsensusVLPs. In some embodiments, the composition contains from about 10 μg to about 100 μg of the Norovirus genotype GI.1 Norwalk VLPs, from about 30 μg to about 200 μg of the Norovirus genotype GII.4 Consensus VLPs, and from about 300 μg to about 700 μg of the adjuvant. In some embodiments, the composition contains either about 15 μg of Norovirus genotype GI.1 Norwalk VLPs and about 50 μg of Norovirus genotype GII.4 ConsensusVLPs or about 50 μg of Norovirus genotype GI.1 NorwalkVLPs and about 150 μg of Norovirus genotype GII.4 ConsensusVLPs, and wherein the composition further contains about 500 μg of aluminum as aluminum hydroxide as adjuvant. In some embodiments, the adjuvant is an aluminum salt. In some embodiments, the aqueous suspension further comprises a sugar selected from the group consisting of trehalose and sucrose. In some embodiments, the aqueous suspension has a sugar content of at least 2% (w/v) or 2.4% (w/v) with respect to the total volume of said suspension. In some embodiments, the aqueous suspension has a content of trehalose of about 4% (w/v) and, optionally 0.4% (w/v) of sucrose, or a content of sucrose of at least 4% (w/v) or at least 4.4% (w/v). In some embodiments, the first frozen suspension in step 2 is formed as a film having a thickness of less than about 500 μm. In some embodiments, said film is formed by contacting the aqueous suspension with a moving cryogenic surface. In some embodiments, the freezing rate in step (a) is ranges from about 50 K/s to about 1100 K/s. In some embodiments, the further drying conditions in step (d) include freeze-drying the second frozen suspension over a period of about 20 to 100 hours and a temperature profile ranging from about −50° C. to about 30° C. at a pressure of less than about 500 mTorr. In some embodiments, the antigen particles have a relative potency of at least 50%, determined by measuring the potency of the antigen particles before and after the composition is dried and based on the potency before the composition is dried using an in vitro relative potency (IVRP) assay. In some embodiments, the antigen particles continue to have a relative potency of at least 50% after storage at 40° C. and 75% relative humidity (RH) for 4 weeks. In some embodiments, the antigen particles have a cumulative undersize distribution with an X₉₀ value of less than about 30 μm, as determined by laser diffraction. In some embodiments, the antigen particles have a relative potency of at least 50%, wherein said relative potency is determined by measuring the potency of the antigens in the antigen particles of a reference aqueous suspension of the composition which has not been subjected to drying and the potency of the antigens in the antigen particles of the solid composition, based on the potency of said reference aqueous suspension.

### Solid Composition Obtainable by the Freeze-Drying Method

According to a third aspect, the present invention is directed to a solid composition obtainable by the freeze-drying method according to the second aspect described herein.

### Glass Vial Containing a Solid Form of a Norovirus Vaccine

According to a fourth aspect, the present invention is directed to a glass vial containing a dose of a Norovirus vaccine in solid form, such as a solid composition according to the first aspect of the present invention, obtainable by applying said dose in form of an aqueous suspension to a wall of the vial cooled to a temperature of below 0° C., e.g. the temperature of liquid nitrogen, allowing the suspension to freeze, and drying the suspension in the vial. In particular, a temperature difference of said vaccine prior to applying to the wall and the temperature of said wall is at least 30° C.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Materials and Methods

### Materials

The Norovirus vaccine used herein was a bivalent virus like particle (VLP) vaccine and comprised Norovirus genotype GI.1 NorwalkVLPs (for sequence of corresponding capsid protein see SEQ ID NO: 2) and genotype GII.4 ConsensusVLPs (for sequence of corresponding capsid protein see SEQ ID NO: 1) adsorbed on aluminum(oxy)hydroxide as part of drug substance manufacturing to generate two adsorbed monovalent bulk drug substances (AMBDS). For production of the vaccine, reference is also made to WO 2010/017542, the entire contents of which are incoporated by reference herein. The two AMBDSs (i.e. GI.1 AMBDS and GII.4 AMBDS) were aseptically blended with additional aluminum(oxy)hydroxide and L-histidine to targeted concentrations of each VLP to create the bivalent vaccine product. The final formulation composition of the drug product was 50/150/500 μg of GI. 1/GII.4/aluminum(oxy)hydroxide per 0.5 mL dose. The bulk drug product also contained 1.55 mg of L-histidine, 4.38 mg of NaCl, 1.73 mg of HCl, and 6.11 mg of sucrose at pH 6.6-7.0. Sucrose (low in endotoxins, suitable for use as excipient EMPROVE® exp Ph Eur, BP, JP, NF) and methanol anhydrate were from Sigma-Aldrich (St. Louis, MO). Trehalose dihydrate (USP grade) was from Pfanstiehl (Waukegan, IL). Aluminum pouches were from IMPAK (Los Angeles, CA). Desiccant was from W. A. Hammond Drierite (Xenia, OH). Silanized R20 glass vials were from Schott (Mainz, Germany).

### Preparation of Vaccine Formulations for Thin-Film Freeze-Drying

The Norovirus vaccine candidate was mixed with a sucrose or trehalose stock solution (50% w/v) in water to reach a final sucrose or trehalose concentration of 0-5% (w/v).

50% of trehalose stock solution and 50% of sucrose stock solution are prepared to produce the final suspension. The contents included in the final suspension are shown in Table 2 below.

TABLE 2
Final composition of the supsensions to reach a final
sucrose or trehalose concentration of 0-5% (w/v).

| | | | Sugar | | | | Mass ratio | |
|---|---|---|---|---|---|---|---|---|
| | | | stock | | | | antigen | Dry |
| | | Norovirus | solution | | | | particles | content**/ |
| Formulations | | vaccine* | 50% | | diH2O | | to sugar | wt.-% |
| Trehalose | 5% Trehalose | 10 mL | 1.11 | mL | 0 | mL | 2.25 × 10⁻² | 78.82 |
| | 4% Trehalose | 10 mL | 889 | μL | 222 | μL | 2.68 × 10⁻² | 75.75 |
| | 3% Trehalose | 10 mL | 667 | μL | 444 | μL | 3.32 × 10⁻² | 71.63 |
| | 2% Trehalose | 10 mL | 444 | μL | 667 | μL | 4.35 × 10⁻² | 65.84 |
| | 1% Trehalose | 10 mL | 222 | μL | 889 | μL | 6.3 × 10⁻² | 57.06 |
| | 0% Trehalose | 10 mL | 0 | mL | 0 | mL | 1.15 × 10⁻¹ | 42.23 |
| Sucrose | 5% Sucrose | 10 mL | 1.11 | mL | 0 | mL | 2.25 × 10⁻² | 78.82 |
| | 4% Sucrose | 10 mL | 889 | μL | 222 | μL | 2.68 × 10⁻² | 75.75 |
| | 3% Sucrose | 10 mL | 667 | μL | 444 | μL | 3.32 × 10⁻² | 71.63 |
| | 2% Sucrose | 10 mL | 444 | μL | 667 | μL | 4.35 × 10⁻² | 65.84 |
| | 1% Sucrose | 10 mL | 222 | μL | 889 | μL | 6.3 × 10⁻² | 57.06 |
| | 0% Sucrose | 10 mL | 0 | mL | 0 | mL | 1.15 × 10⁻¹ | 42.23 |

*Each vaccine suspension for preparation of the final formulation and before it is mixed with the stock solution includes additional 1.22% (w/v) of sucrose which is not considered for the indications “5% sucrose/trehalose, 4% sucrose/trehalose, 3% sucrose/trehalose, 2% sucrose/trehalose, 1% sucrose/trehalose and 0% sucrose/trehalose”.
**Calculated based on the moisture-free form of the lyophilizate after TFFD.

For single-vial TFFD, 250 μL was applied in drops to the bottom surface of glass vials (silanized R20 glass vials from Schott (Mainz, Germany).

### Thin-Film Freezing (TFF)

The Norovirus vaccine in suspension containing sucrose or trehalose (0-5%, w/v) was subjected to TFF. Briefly, 1 mL of sample was dropped onto a rotating cryogenically cooled stainless steel surface to form frozen thin-films at a calculated freezing rate of about 100 K/s to about 1000 K/s (the calculation is based on J. D. Engstrom et al., Pharmaceutical Research 2008; 25(6): 1334-1346). In order to avoid the overlap of droplets, the rotating speed at which the cryogenic steel surface of a drum having a diameter of 10 cm, on which the vaccine suspension was dropped, was controlled at 5-7 rpm. The frozen thinfilms were removed by a steel blade and collected in liquid nitrogen in a salinized glass. The glass vial was capped with rubber stopper with half open (i.e. the vial was not fully capped, leaving space between the mouth of the vial and the legs of the rubber stopper for water molecules to escape and for nitrogen molecules to enter the vial after the lyophilization step) and then transferred into a VirTis Advantage bench top tray lyophilizer with stopper re-cap function (The VirTis Company, Inc. Gardiner, NY).

For single-vial TFF, a R20 salinized glass vial was immersed into liquid nitrogen for 10 min to create a cryogenically cooled surface in the inner bottom of the vial. A syringe with a 18G1 needle was used to add 250 μL of the Norovirus vaccine candidate in suspension dropwise to the bottom of the vial so that the droplets, upon impact of the surface, rapidly froze into thin-films. The vial was capped with a rubber stopper with half open and then transferred to the lyophilizer as mentioned above for lyophilization.

### Lyophilization and Packaging

Lyophilization was performed over 60 h at pressures less than 200 mTorr, while the shelf temperature was gradually ramped from −40° C. to 25° C. The lyophilization cycle is shown in Table 3. After lyophilization, vacuum was released, and the lyophilizer was filled with nitrogen gas. Each glass vial was tightly capped with a rubber stopper using the automatic stopper-recap function in the lyophilizer and sealed with an aluminum cap. The vial was then placed individually into an aluminum pouch with with desiccant (Drierite desiccant bag or molecular sieve, 1 Qz) inside. The pouch was sealed and then stored at 4° C. before further use.

TABLE 3
Lyophilization cycle used to lyophilize
the thin-film frozen Noro vaccine.

| Lyophilization Stage | Parameters | |
|---|---|---|
| Loading/Freezing temp | −40° | C. |
| Primary drying temp | −40° | C. |
| Primary drying time | 20 | h |
| Ramp to secondary drying | 20 | h |
| Secondary drying temp | +25° | C. |
| Secondary drying time | 20 | h |

### Characterization of the Thin-Film Freeze-Dried Norovirus Vaccine

The moisture content in the dried powder was determined using a Karl Fisher Titrator Aquapal III from CSC Scientific Company (Fairfax, VA). To determine the particle size and size distribution, vials with thin-film freeze-dried Norovirus vaccine powder were randomly selected, and the powder was reconstituted with water (1 mL for bulk TFFD, and 250 μL for single-vial TFFD). The particle size and size distribution in the reconstituted Norovirus vaccine, after 2-fold dilution in order to reach an obscuration effect of ˜10%, were determined using a Sympatec HELOS laser diffraction instrument equipped with a R3 lens (Sympatec GmbH, Germany). The pH value of the reconstituted Norovirus vaccine was measured using a Mettler Toledo's SevenCompact pH meter S220. The concentration of the GI.1 NV-VLP and G II.4 C-VLP antigens in the reconstituted vaccine after desorption was determined by RP-HPLC with an Agilent 1260. The column used was an Agilent PoroShell 300SB-C8 2.1×75 mm, 5 μm.

### In Vitro Relative Potency (IVRP) Assay

The potency of the antigens before and after the vaccine was subjected to TFFD was determined using an in vitro relative potency (IVRP) assay. In the IVRP assay, monoclonal antibodies (mAbs) directed to either the Consensus GII.4 or Norwalk GI.1 VLP are incubated with the vaccine samples first to allow binding of the mAbs to the vaccine samples. Afterwards, remaining (i.e. unbound) mAbs are determined in an enzyme-linked immunosorbent assay (ELISA) set-up. Remaining (i.e. unbound) mAbs are indicative for the amount of intact epitopes in the corresponding vaccines samples (i.e. epitopes that still can be bound by the corresponding mAb) and thus for the potency of the samples.

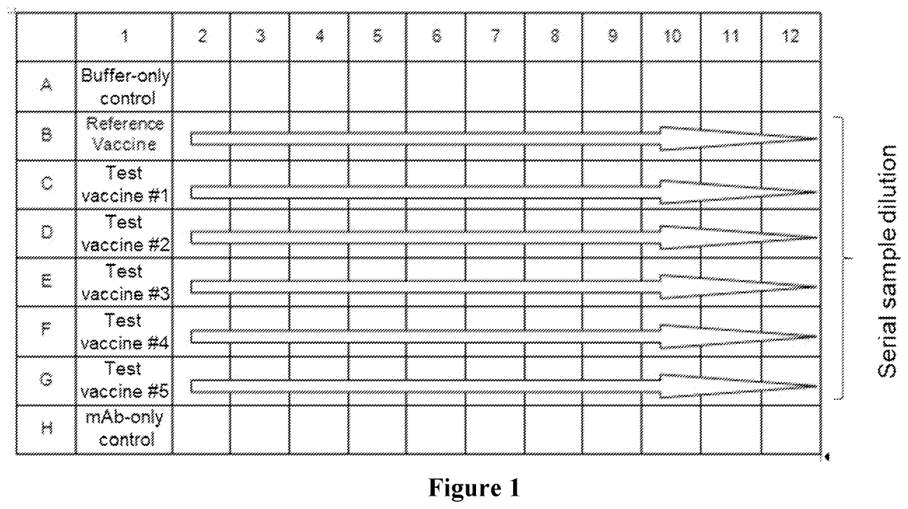

In a first step, the test vaccine (i.e. the vaccine composition which was not subjected to TFFD and the reconstituted, with water to achieve a concentration of 100 μg/ml, solid vaccine composition after TFFD was carried out, e.g. at 0 weeks and at 4 weeks under accelerated conditions), as well as a reference vaccine, were serially diluted (from 8.333 to 0.001 μg/mL of GII.4 Consensus VLP) in a dilution plate (Nunc™ 96-Well Polypropylene Storage Microplates, Thermo Scientific, Cat. #24994) using blocking buffer (1X phosphate buffered saline, pH 7.4 (PBS)+0.05% Tween-20 (Fisher Scientific,Cat #BP337)+10% goat serum (Sterile Filtered Goat Serum, Equitech-Bio, Cat. #SG30)) to result in a volume of 150 μL per well. The reference vaccine comprised 50 μg of GI. 1 Norwalk VLP and 150 μg of GII.4 Consensus VLP adsorbed onto 500 μg aluminum hydroxide (50/150/500 μg) per 0.5 mL and was not subjected to TFFD. The reference vaccine was kept at 2-8° C. prior to use and mixed until the material was homogeneous (free of precipitates) by visual inspection prior to application in the IVRP assay. After dilution, either a mAb binding to Consensus GII.4 VLPs (BioGenes; IgG clone #1-9-1; 8.0 mg/mL stock concentration, Lot-No. PP120514-001) or a mAb binding to Norwalk GI.1 VLPs was added to allow binding of the mAbs to the reference vaccine or test vaccine, respectively. Therefore, the mAbs were diluted in blocking buffer to a final concentration of 0.08 μg/mL and subsequently, 50 μL of the diluted mAbs were added per well to result in a total volume of 200 μL per well. In addition, a buffer-only control row, solely comprising 200 μL blocking buffer per well and a mAb-only control row, comprising 150 μL blocking buffer mixed with 50 μL mAb per well were included. For an exemplarily plate layout, reference is made to FIG. 1. Three replicates, i.e. three identical dilution plates were prepared. The dilution plates were sealed with aluminum plate sealer and placed in a humidified incubator (Thermo Scientific, ≥85% relative humidity, 5% CO₂) at 37° C.±2° C. for 220±10 minutes.

During incubation of the dilution plates, assay plates were coated with either Consensus GII.4 VLP or Norwalk GI. 1 reference VLPs. Therefore, a coating solution for each VLP was prepared by dilution of the VLP to 5 μg/mL in 1×PBS. Then, 100 μL of the corresponding coating solutions were added per well into flat bottom 96-well plates. The assay plates were sealed and incubated at room temperature for 130+10 minutes. After incubation, the assay plates were washed three times with 350 μL/well of wash buffer (1X PBS+0.05% Tween-20) using a Molecular Devices' AquaMax 4000 and subsequently blocked by adding 150 μL/well of blocking buffer. The plates were sealed and incubated at room temperature for 80±20 minutes. After incubation, the assay plates were again washed three times with 350 μL/well of wash buffer (1X PBS+0.05% Tween-20).

After incubation, the content of each well of the dilution plates was mixed by pipetting up and down and subsequently 100 μL were transferred per well to the assay plates. The plate layout of the dilution plates was thereby retained for the assay plates (see, for instance, FIG. 1). The assay plates were sealed and incubated at room temperature for 60±5 minutes. After incubation, the assay plates were washed three times with 350 μL/well of wash buffer (1X PBS+0.05% Tween-20).

In a next step, secondary antibody solution was prepared. Therefore, goat anti-mouse IgGI horseradish peroxidase (HRP) antibody (stored at 2-8° C.; Southern Biotech, Cat.-No. 1070-05) was diluted in blocking buffer to a final concentration of 0.292 μg/mL. 100 μL of the prepared secondary antibody solution were added per well to the assay plates. Afterwards, the assay plates were sealed and incubated at room temperature for 30+10 minutes. After incubation, the assay plates were washed three times with 350 μL/well of wash buffer (1X PBS+0.05% Tween-20). Then, 100 μL of 2,2′-azino-bis-3-ethylbenzothiazoline-6-sulphonic acid (ABTS) peroxidase subtrate solution (1-Component™ ABTS Substrate Solution, Thermo Scientific, Cat. #37615) were added to each well. The plates were incubated for 20±2 minutes. After incubation, 100 μL of stop solution (Seracare Life Sciences ABTS HRP Stop Solution, Fisher Scientific, Cat. #51500017) were added to each well. Within 1 hour of the addition of stop solution, absorbance at 405 nm was read in a plate reader (Molecular Devices' SpectraMax i3x).

The absorbance values for the test vaccines and reference vaccine were substracted by the median absorbance values from the buffer-only control and subsequently were plotted in dependency of the vaccine dilution. The curves were fit using an independent 4-Paramter Logistic (4-PL) fit and dilutions referring to 50% of maximal absorbance signal were determined (EC50 values or inflection points, respectively). The relative potency of the test vaccine was subsequently determined for each VLP by dividing the inflection point of the test vaccine by the inflection point of the reference vaccine. Average relative potencies across the three replicates were calculated. As acceptance criteria, inter alia the curve fit (R2-value) for the reference vaccine from the independent 4-parameter logistic fit must be higher than 0.980 and the average optical density at 405 nm for the mAb-only control wells (positive controls; mAb binding in the ELISA is not reduced due to pre-incubation with a vaccine sample) must be higher than 1.3.

### Accelerated Stability Study

Randomly selected vials with thin-film freeze-dried Norovirus vaccine powder were stored at 40° C., 75% relative humidity (RH), for 4 weeks, and the potency of the antigens upon reconstitution was determined using the IVRP assay as mentioned above.

### Statistical Analysis

Statistical analyses were conducted using analysis of variance (ANOVA) followed by Fisher's protected least significant difference procedure. A p-value of ≤0.05 (two-tail) was considered significant.

### Example 1: Effect of Concentration of Trehalose or Sucrose on Particle Size and Size Distribution of the Norovirus Vaccine Candidate after it was Subjected to TFFD and Reconstitution

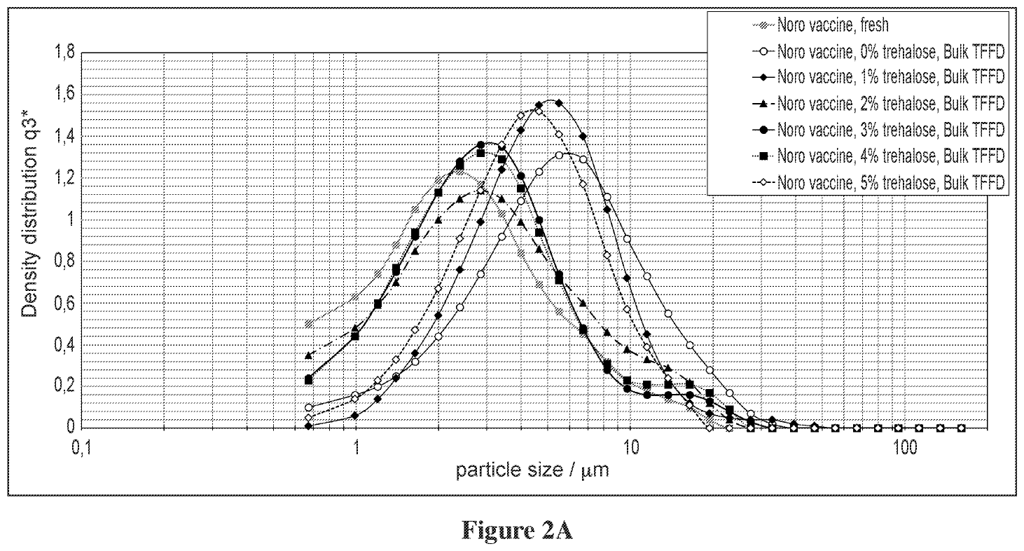

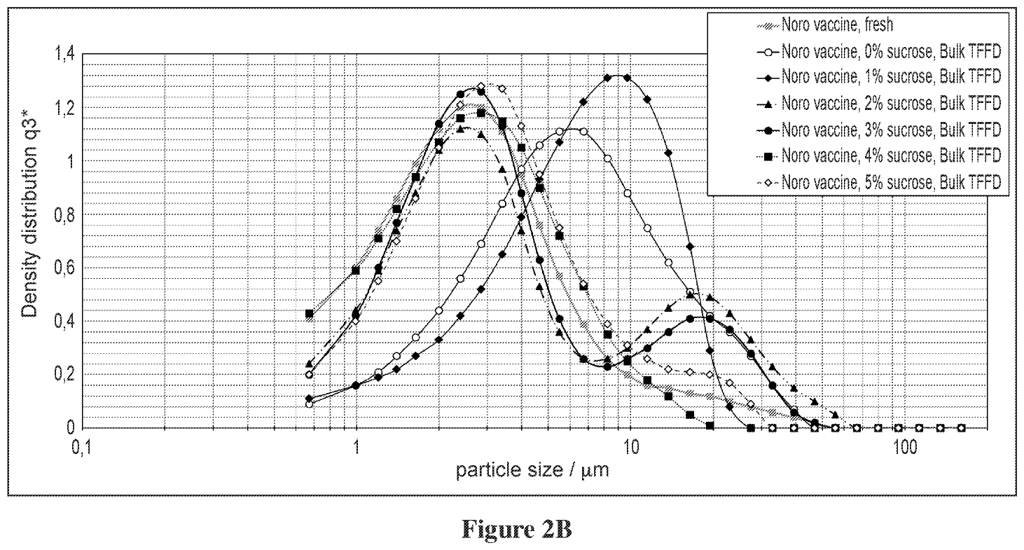

When measuring particles size and size distribution in the Norovirus vaccine, to confirm consistency and reproducibility in the readings, the obscuration value on the particle size data was studied first by determining the particle size and size distribution after the original Norovirus vaccine was diluted from 0-fold to 20-fold in water. It was concluded that the vaccine needed to be diluted two-fold to reach an optimum obscuration level of no more than 10-15%, based on the recommended range for material size measurement (see Kulkarni VS, Shaw C. Chapter 8-Particle Size Analysis: An Overview of Commonly Applied Methods for Drug Materials and Products. In: Kulkarni VS, Shaw C, editors. Essential Chemistry for Formulators of Semisolid and Liquid Dosages. Boston: Academic Press; 2016. p. 137-44). Shown in FIG. 2A is the effect of concentration of trehalose on particle size and size distribution after the Norovirus vaccine was subjected to bulk TFFD and reconstitution, while FIG. 2B shows the effect of concentration of sucrose on particle size and size distribution after the Norovirus vaccine was subjected to bulk TFFD and reconstitution. The results indicated that 2-4% (w/v) of trehalose or 2-5% (w/v) of sucrose were advantageous to help maintain particle size and size distribution in the Norovirus vaccine after it was subjected to TFFD, because these formulations showed less particle aggregation after reconstitution. The results for 4% (w/v) trehalose and 4-5% (w/v) sucrose showed the least particle aggregation.

### Example 2: Effect of Bulk TFFD Vs. Single-Vial TFFD on Particle Size and Size Distribution of the Norovirus Vaccine

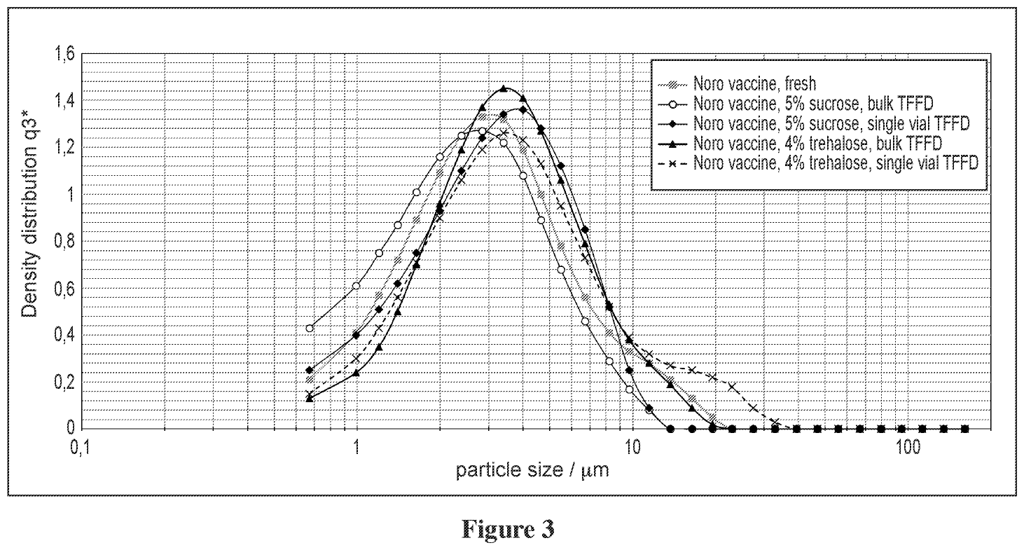

To understand the effect of the bulk TFF process compared to the single-vial TFF process on the particle size and size distribution of Norovirus vaccine after TFFD, Norovirus vaccine suspended in 4% trehalose or 5% sucrose was subjected to single-vial TFFD or bulk TFFD. Shown in FIG. 3 and Table 4 are representative particle size and size distribution after the dry powders were reconstituted.

TABLE 4
Representative particle size and size distribution of
bulk and single vial-TFFD of Noro vaccine in 4% concentration
of trehalose, and 5% concentration of sucrose.

| Formulations | X₁₀ (μm) | X₅₀ (μm) | X₉₀ (μm) |
|---|---|---|---|
| Noro vaccine, fresh | 1.02 ± 0.19 | 2.71 ± 0.33 | 7.67 ± 0.17 |
| Noro vaccine, 5% sucrose, | 1.13 ± 0.21 | 2.62 ± 0.42 | 8.65 ± 2.74 |
| bulk TFFD | | | |
| Noro vaccine, 5% sucrose, | 0.99 ± 0.16 | 2.85 ± 0.45 | 6.76 ± 0.13* |
| single vial TFFD | | | |
| Noro vaccine, 4% trehalose, | 1.62 ± 0.26 | 3.82 ± 0.57 | 8.43 ± 0.67 |
| bulk TFFD | | | |
| Noro vaccine, 4% trehalose, | 1.52 ± 0.27 | 3.84 ± 0.45 | 8.86 ± 2.72 |
| single vial TFFD | | | |

X₁₀, X₅₀, X₉₀ denote particle dimensions corresponding to 10%, 50% and 90% of the cumulative undersize distribution. Data are mean ± SD (n = 2 or 3. *p < 0.05, Norovirus vaccine vs. fresh).

The residual moisture content in the thin-film freeze-dried Norovirus vaccine powders were 1.5-1.8% (Table 5), well below the usually suggested 3% limit.

TABLE 5
Characterizations in TFFD Norovirus vaccine
powder and the vaccine after reconstitution.
Data are mean ± S.D. (n = 2 or 3).

| | Before | | |
|---|---|---|---|
| | reconstitution | | |
| | Residual moisture | After reconstitution | |
| Formulations | content* | Osmolality+ | pH+ |
| Freshly prepared Noro vaccine | NA | 310 | 6.56 |
| as shipping control | | | |
| Noro vaccine, 5% sucrose, | 1.80 ± 0.15% | 304 | 6.48 |
| bulk TFFD | | | |
| Noro vaccine, 5% sucrose, | 1.66 ± 0.11% | 517 | 6.37 |
| single vial TFFD | | | |
| Noro vaccine, 4% trehalose, | 1.57 ± 0.17% | 289 | 6.43 |
| bulk TFFD | | | |
| Noro vaccine, 4% trehalose, | 1.48 ± 0.15% | 567 | 6.45 |
| single vial TFFD | | | |

*Moisture content for single vial-TFFD powder was measured in two vials.
+Osmolality and PH was measured in one vial.

### Example 3: Characterization of Thin-Film Freeze-Dried Norovirus Vaccine Upon Reconstitution

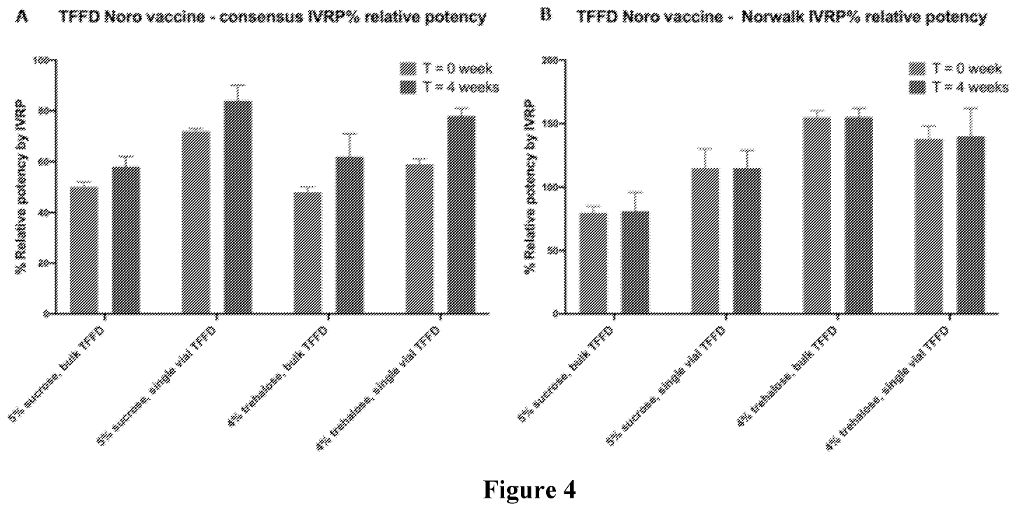

Upon reconstituting the Norovirus vaccine dry powders prepared with 4% (w/v) of trehalose or 5% (w/v) of sucrose by either single-vial TFFD or bulk TFFD, we also measured the pH, antigen concentration, and antigen potency in the reconstituted Norovirus vaccine suspension. The pH values of the vaccine remained 6.4-6.5 (see Table 4). The recovery rate of the Consensus antigen was 103±16%, 104±15% for the Norwalk antigen, as determined by RP-HPLC. The relative potency as measured by the IVRP assay for the Consensus antigen was between 49-73%, and 81-154% for the Norwalk antigen (FIGS. 4A and 4B), which meets an acceptable range of 50-150%.

### Example 4: Potency of the Norovirus Vaccine Powders after Four Weeks of Storage at 40° C., 75% Relative Humidity

In an accelerated stability study, the Norovirus vaccine dry powder was stored at 40° C., 75% RH, for 4 weeks, and then the antigen potency using the IVRP assay was measured. As shown in FIGS. 4A and 4B, the potency of both GII.4 Consensus and GI.1 Norwalk antigens did not change significantly.

What is claimed is:

1. A solid composition comprising:
antigen particles comprising virus like particles (VLPs) adsorbed on adjuvant particles, wherein the VLPs are Norovirus VLPs.

2. The solid composition according to claim 1, wherein the antigen particles have a cumulative undersize distribution with an X₉₀ value of less than about 30 μm, as determined by laser diffraction.

3. The solid composition according to claim 1 or 2, wherein the antigen particles have a relative potency of at least 50%, wherein said relative potency is determined by measuring the potency of the antigens in the antigen particles of a reference aqueous suspension of the composition which has not been subjected to drying and the potency of the antigens in the antigen particles of the solid composition, based on the potency of said reference aqueous suspension.

4. The solid composition according to claim 3, wherein the antigen particles continue to have a relative potency of at least about 50% after storage at 40° C. and 75% relative humidity (RH) for 4 weeks.

5. The solid composition according to any one of claims 1 to 4 further comprising a sugar selected from the group consisting of trehalose, lactose and sucrose; or further comprising a sugar alcohol.

6. The solid composition according to claim 5, wherein a mass ratio between the antigen particles and the sugar, based on the mass of the sugar in the solid composition, is about 4.2×10⁻² or less; and/or wherein a dry content of the sugar is at least about 60.00 wt.-%, wherein the dry content is calculated based on the moisture-free solid composition.

7. The solid composition according to claim 6, wherein the mass ratio between the antigen particles and the mass of the sugar is about 2.68×10⁻²; the mass ratio between the antigen particles and sucrose is about 2.8×10⁻² or less, and/or the dry combined contents of trehalose and sucrose is about 75.75 wt.-%, or the dry content of sucrose is at least about 75.75 wt.-%.

8. The solid composition according to claim 5, wherein the composition is obtained from an aqueous solution comprising 2-4% w/v trehalose and/or 2-5% w/v sucrose.

9. The solid composition according to any one of claims 1 to 8, wherein the composition comprises one or more different genogroups of Norovirus VLPs adsorbed on the adjuvant, wherein said genogroups are selected from the group consisting of Norovirus genogroup 1 (GI) VLP and Norovirus genogroup 2 (GII) VLPs.

10. The solid composition according to claim 9, wherein the VLPs consist of Norovirus genotype GI.1 NorwalkVLPs and Norovirus genotype GII.4 ConsensusVLPs.

11. The solid composition according to claim 10, containing from about 10 μg to about 100 μg of the Norovirus genotype GI.1 NorwalkVLPs, from about 30 μg to about 200 μg of the Norovirus genotype GII.4 ConsensusVLPs, and from about 300 μg to about 700 μg of the adjuvant.

12. The solid composition according to claim 10 or 11, wherein the VLPs consist of the Norovirus genotype GI.1 NorwalkVLPs and the Norovirus genotype GII.4 ConsensusVLPs, wherein the composition contains either about 15 μg of Norovirus genotype GI.1 NorwalkVLPs and about 50 μg of Norovirus genotype GII.4 ConsensusVLPs or about 50 μg of Norovirus genotype GI.1 NorwalkVLPs and about 150 μg of Norovirus genotype GII.4 ConsensusVLPs, and wherein the composition further contains about 500 μg of aluminum as aluminum hydroxide as adjuvant.

13. The solid composition according to any one of claims 1 to 11, wherein the adjuvant is an aluminum salt.

14. The solid composition according to any one of claims 1 to 13, wherein the composition is an immunogenic composition.

15. The solid composition according to claim 14, wherein the immunogenic composition is a vaccine composition.

16. A solid composition obtained by a freeze-drying method for removing water from an aqueous suspension of antigen particles comprising virus like particles (VLPs) adsorbed on adjuvant particles, wherein the VLPs are Norovirus VLPs, the method comprising the steps of:
a) providing the aqueous suspension, optionally at a temperature ranging from about 2° C. to about 30° C.;
b) decreasing the temperature of step (a) with a freezing rate of at least 50 K/s to obtain a first frozen suspension;
c) collecting said first frozen suspension in a container cooled to the temperature of liquid nitrogen to obtain a second frozen suspension;
d) subjecting said second frozen suspension to further drying conditions under reduced pressure to obtain a solid composition.

17. A glass vial containing a single dose of a Norovirus vaccine in solid form, such as a solid composition according to any one of claims 1 to 15, obtainable by applying said single dose in form of an aqueous suspension to a wall of the vial cooled to a temperature of liquid nitrogen, allowing the suspension to freeze, and drying the suspension in the vial.

18. A freeze-drying method for removing water from an aqueous suspension of antigen particles comprising virus like particles (VLPs) adsorbed on adjuvant particles, wherein the VLPs are Norovirus VLPs, the method comprising the steps of:
a) providing the aqueous suspension, optionally at a temperature ranging from about 2° C. to about 30° C.;
b) decreasing the temperature of step (a) with a freezing rate of at least 50 K/s to obtain a first frozen suspension;
c) collecting said first frozen suspension in a container cooled to the temperature of liquid nitrogen to obtain a second frozen suspension;
d) subjecting said second frozen suspension to further drying conditions under reduced pressure to obtain a solid composition according to claim 1.

19. The freeze-drying method according to claim 18, wherein the composition comprises one or more different genogroups of Norovirus VLPs selected from the group consisting of Norovirus genogroup 1 (GI) VLPs and Norovirus genogroup 2 (GII) VLPs.

20. The freeze-drying method according to claim 19, wherein the VLPs consist of Norovirus genotype GI.1 NorwalkVLPs and Norovirus genotype GII.4 ConsensusVLPs.

21. The freeze-drying method according to claim 20, wherein the composition contains from about 10 μg to about 100 μg of the Norovirus genotype GI.1 NorwalkVLPs, from about 30 μg to about 200 μg of the Norovirus genotype GII.4 ConsensusVLPs, and from about 300 μg to about 700 μg of the adjuvant.

22. The freeze-drying method according to claim 20 or 21, wherein the composition contains either about 15 μg of Norovirus genotype GI.1 NorwalkVLPs and about 50 μg of Norovirus genotype GII.4 ConsensusVLPs or about 50 μg of Norovirus genotype GI.1 NorwalkVLPs and about 150 μg of Norovirus genotype GII.4 ConsensusVLPs, and wherein the composition further contains about 500 μg of aluminum as aluminum hydroxide as adjuvant.

23. The freeze-drying method according to any one of claims 18 to 21, wherein the adjuvant is an aluminum salt.

24. The freeze-drying method according to any one of claims 18 to 23, wherein the aqueous suspension further comprises a sugar selected from the group consisting of trehalose and sucrose.

25. The freeze-drying method according to any one of claims 18 to 24, wherein the aqueous suspension has a sugar content of at least 2% (w/v) or 2.4% (w/v) with respect to the total volume of said suspension.

26. The freeze-drying method according to any one of claims 18 to 25, wherein the aqueous suspension has a content of trehalose of about 4% (w/v) and, optionally 0.4% (w/v) of sucrose, or a content of sucrose of at least 4% (w/v) or at least 4.4% (w/v).

27. The freeze-drying method according to any one of claims 18 to 26, wherein the first frozen suspension in step (b) is formed as a film having a thickness of less than about 500 μm.

28. The freeze-drying method according to claim 27, wherein said film is formed by contacting the aqueous suspension with a moving cryogenic surface.

29. The freeze-drying method according to any one of claims 18 to 28, wherein the freezing rate in step (a) ranges from about 50 K/s to about 1100 K/s.

30. The freeze-drying method according to any one of claims 18 to 29, wherein the further drying conditions in step (d) include freeze-drying the second frozen suspension over a period of about 20 to 100 hours and a temperature profile ranging from about −50° C. to about 30° C. at a pressure of less than about 500 mTorr.

31. The freeze-drying method according to any one of claims 18 to 30, wherein the antigen particles have a relative potency of at least 50%, determined by measuring the potency of the antigen particles before and after the composition is dried and based on the potency before the composition is dried using an in vitro relative potency (IVRP) assay.

32. The freeze-drying method according to claim 31, wherein the antigen particles continue to have a relative potency of at least 50% after storage at 40° C. and 75% relative humidity (RH) for 4 weeks.

33. The freeze-drying method according to any one of claims 18 to 32, wherein the antigen particles have a cumulative undersize distribution with an X₉₀ value of less than about 30 μm, as determined by laser diffraction.

34. The freeze-drying method according to any one of claims 18 to 33, wherein the antigen particles have a relative potency of at least 50%, wherein said relative potency is determined by measuring the potency of the antigens in the antigen particles of a reference aqueous suspension of the composition which has not been subjected to drying and the potency of the antigens in the antigen particles of the solid composition, based on the potency of said reference aqueous suspension.

35. A solid composition obtainable by the freeze-drying method according to any one of claims 18 to 34.


SEQUENCE LISTING (3 sequences)

SEQ ID NO: 1 - protein, 540 aa, Artificial Sequence
  Composite GII.4 Norovirus VP1 amino acid sequence
MKMASSDANPSDGSTANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDPTIKLIAMLYTPLRANNAGDDVFTVSCRVLTRPSPDFDFIFLVPPTVESRTKPFTVPILTVEEMTNSRFPIPLEKLFTGPSGAFVVQPQNGRCTTDGVLLGTTQLSPVNICTFRGDVTHIAGTQEYTMNLASQNWNNYDPTEEIPAPLGTPDFVGKIQGVLTQTTRGDGSTRGHKATVSTGSVHFTPKLGSVQFSTDTSNDFETGQNTKFTPVGVVQDGSTTHQNEPQQWVLPDYSGRDSHNVHLAPAVAPTFPGEQLLFFRSTMPGCSGYPNMNLDCLLPQEWVQHFYQEAAPAQSDVALLRFVNPDTGRVLFECKLHKSGYVTVAHTGQHDLVIPPNGYFRFDSWVNQFYTLAPMGNGTGRRRAL

SEQ ID NO: 2 - protein, 530 aa, Unknown
  Norovirus Norwalk virus sp.
MMMASKDATSSVDGASGAGQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGNAFTAGKIIVSCIPPGFGSHNLTIAQATLFPHVIADVRTLDPIEVPLEDVRNVLFHNNDRNQQTMRLVCMLYTPLRTGGGTGDSFVVAGRVMTCPSPDFNFLFLVPPTVEQKTRPFTLPNLPLSSLSNSRAPLPISSIGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTASSARGRLGLRR

SEQ ID NO: 3 - protein, 530 aa, Unknown
  Norovirus Norwalk virus sp.
MMMASKDATSSVDGASGAGQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGNAFTAGKIIVSCIPPGFGSHNLTIAQATLFPHVIADVRTLDPIEVPLEDVRNVLFHNNDRNQQTMRLVCMLYTPLRTGGGTGDSFVVAGRVMTCPSPDFNFLFLVPPTVEQKTRPFTLPNLPLSSLSNSRAPLPISSMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTASSARGRLGLRR